(19) United States
(12) Patent Application Publication
(10) Pub. No.: US 2026/0294233 A1
(43) Pub. Date: Oct. 1, 2026

(54) SYSTEMS AND METHODS FOR TESTING AND/OR MONITORING VISION

(71) Applicant: Tilak Healthcare SAS, Paris (FR)

(72) Inventors: Hélène STARYNKEVITCH, Versailles (FR); Audrey THIREL, Paris (FR)

(73) Assignee: Tilak Healthcare SAS, Paris (FR)

(21) Appl. No.: 19/574,065

(22) Filed: Mar. 20, 2026

Related U.S. Application Data
(60) Provisional application No. 63/784,862, filed on Apr. 7, 2025.

(30) Foreign Application Priority Data
Mar. 31, 2025 (EP) 25305469.6

(51) Int. Cl. A61B 3/032; A61B 3/00; A63F 13/52; A63F 13/798; G16H 50/20

(52) U.S. Cl. CPC A61B 3/032; A61B 3/0025; A61B 3/0033; A61B 3/0041; G16H 50/20; A63F 13/52; A63F 13/798

(57) ABSTRACT

Systems and methods are provided herein for testing vision of a patient, monitoring vision of a patient, and/or determining changes in vision. The vision system may include multiple types of vision tests that a patient may access on their mobile device. For example, a patient may perform acuity tests including optotypes and may also use an Amsler grid to indicate the presence of one or more scotoma and/or metamorphopsia in the patient's field of vision. The results from the acuity tests and Amsler grids may be used to manipulate access to a video game, also presented on the patient's mobile device. For example, access to certain levels and/or game play may be manipulated. Visual metrics may be determined based on the results from the Amsler grid and/or acuity testing, and optionally the video game.

### CROSS-REFERENCE TO RELATED APPLICATIONS

This application claims priority to U.S. Provisional Patent Application No. 63/784,862, filed Apr. 7, 2025, and European Patent Application No. 25305469.6, filed Mar. 31, 2025, the entire contents of each of which are incorporated herein by reference.

### TECHNICAL FIELD

This technology relates, in general, to a system for testing, monitoring and/or estimating a change in vision and/or vision loss in a patient.

### BACKGROUND

In the United States alone, millions of people suffer from eye disease and/or have vision impairment. It is estimated that around 2 million people in the United States have advanced age-related macular degeneration, almost 3 million people in the United States have glaucoma, and almost 8 million people in the United States have diabetic retinopathy.

For patient's with vision and/or eye diseases, regular monitoring of visual parameters is crucial to providing timely treatment and preventing further vision loss. For example, patients with certain eye diseases such as macular diseases often must be routinely monitored and may receive frequent injections (e.g., anti-VEGF injections) to maintain their vision. Patient's with macular disease may average about 6 injections per year to treat and extend their vision.

One significant challenge to patients with vision and/or eye disease is that there is a shortage of ophthalmologists and other trained health care providers, with only about 5.9 ophthalmologists per 100,000 people in the United States, and the number of patients is expected to increase in the coming years. Remote monitoring of visual acuity by a healthcare provider such as an ophthalmologists can help reduce the overall costs and the burden on patients and caregivers while preserving vision, however remote monitoring by ophthalmologists remains difficult given the limited number of ophthalmologists and trained healthcare providers. Further techniques for remotely monitoring evolution of a pathology are limited.

Accordingly, there is a need for improved methods and systems for monitoring and testing a patient's visions as well as analyzing and/or processing various visual testing data to determine a change in vision and/or evolution of a pathology.

### SUMMARY

Provided herein are systems for testing vision of a patient, monitoring vision of a patient, determining changes in vision, and/or estimating changes in vision. The vision system may include multiple types of vision tests that a patient may access on their mobile device or other electronic device. The results may be collectively analyzed and used to monitor the patient's vision and/or determine or estimate changes in the patient's vision. A patient may perform acuity tests and may also use an Amsler grid to indicate the presence of one or more scotoma and/or metamorphopsia in the patient's field of vision. The results from the acuity tests and Amsler grids may be analyzed to determine visual metrics. The visual metrics may be used to manipulate access to a video game, which also may be presented on the patient's mobile device or other electronic device. The video game may generate data indicative vision metrics of the patient based on the patient's performance in the video game. The visual metrics from the acuity test, Amsler test, and/or video game may be used to generate an alert corresponding to a change in a patient's vision and/or may be used to estimate risk of evolution of a disease.

A method is provided herein for detecting a change in vision of a user. The method may include causing an optotype to be displayed on a device of the user, the optotype oriented in a first direction, receiving acuity results corresponding to the optotype and first user input indicative a second direction associated with the optotype, generating a first visual metric corresponding to the user based on the first direction, the acuity results, and one or more of a distance between the user and the device or a guessing rate, causing an Amsler grid to be displayed on the device, receiving first Amsler results including second user input corresponding to one or more grid markings on the Amsler grid indicative of vision distortions of the user, training a machine learning model to generate at least one visual metric corresponding to Amsler results, generating a second visual metric of the user based on the Amsler results and using the machine learning model, and generating video game metrics corresponding to a video game based on the first visual metric and the second visual metric, the video game metrics corresponding to at least a difficulty level of the video game.

The method may further include generating a third visual metric based on at least the first visual metric and the second visual metric, the third visual metric indicative of the change in vision of the user. The method may further include including generating a third visual metric corresponding to the user based on a user's performance in the video game. The method may further include generating a fourth visual metric corresponding to the user based on the first visual metric, the second visual metric, and the third visual metric, the fourth visual metric indicative of the change in vision of the user. The method may further include generating an alert based on one or more of the first visual metric, the second visual metric, the third visual metric, or the fourth visual metric. The video game may be configured to be displayed on the device. The method may further include determining a test frequency, wherein one or more of the optotype or the Amsler grid is caused to be displayed on the device based on the test frequency. The method may include the guessing rate is based on wrong answers and unknown answers corresponding to a set of previously displayed optotypes. The method may include calculating the distance between the user and the device based on a distance between two landmarks identified on an image of the user generated by the device. The machine learning model may include at least one convolutional neural network (CNN) and the second visual metric comprises one or more of a likelihood of a presence of one or more scotoma and/or one or more metamorphopsia, amount data corresponding to the one or more scotoma and/or the one or more metamorphopsia, and/or size data corresponding to the one or more scotoma and/or the one or more metamorphopsia.

A system for detecting a change in vision of a user is provided herein. The system may include memory configured to store computer-executable instruction, and at least one computer processor configured to access memory and execute the computer-executable instructions to cause an optotype to be displayed on a device of the user, the optotype oriented in a first direction, receive acuity results corresponding to the optotype and first user input indicative a second direction associated with the optotype, generate a first visual metric corresponding to the user based on the first direction, the acuity results, and one or more of a distance between the user and the device or a guessing rate, cause an Amsler grid to be displayed on the device, receive first Amsler results including second user input corresponding to one or more grid markings on the Amsler grid indicative of vision distortions of the user, train a machine learning model to generate at least one visual metric corresponding to Amsler results, generate a second visual metric of the user based on the Amsler results and using the machine learning model, and generate video game metrics corresponding to a video game based on the first visual metric and the second visual metric, the video game metrics corresponding to at least a difficulty level of the video game.

The at least one computer processor may be further configured to access memory and execute the computer-executable instructions to generate a third visual metric corresponding to the user based on a user's performance in the video game. The at least one computer processor may be further configured to access memory and execute the computer-executable instructions to generate a fourth visual metric corresponding to the user based on the first visual metric, the second visual metric, and the third visual metric, the fourth visual metric indicative of the change in vision of the user. The guessing rate may be based on wrong answers and unknown answers corresponding to a set of previously displayed optotypes. The machine learning model comprises at least one convolutional neural network (CNN) and the second visual metric comprises a likelihood of a presence of one or more scotoma and/or one or more metamorphopsia.

Yet another method is provided for detecting a change in a vision of a user. The method may include causing an optotype to be displayed on a device of the user, the optotype oriented in a first direction, receiving acuity results corresponding to the optotype and first user input indicative a second direction associated with the optotype, generating a first visual metric corresponding to the user based on the first direction, the acuity results, and one or more of a distance between the user and the device or a guessing rate, causing an Amsler grid to be displayed on the device of the user, receiving first Amsler results including second user input corresponding to one or more grid markings on the Amsler grid indicative of vision distortions of the user, training a machine learning model to generate at least one visual metric corresponding to Amsler results, generating a second visual metric of the user based on the first Amsler results and using the machine learning model, causing a video game to be displayed on the device, and generating a third visual metric corresponding to the user based on a user's performance in the video game.

The method may further include generating video game metrics corresponding to the video game based on the first visual metric and the second visual metric, the video game metrics corresponding to at least a difficulty level of the video game. The method may further include generating a fourth visual metric corresponding to the user based on the first visual metric, the second visual metric, and the third visual metric, the fourth visual metric indicative of the change in vision of the user. The method is generating an alert based on one or more of the first visual metric, the second visual metric, the third visual metric, or the fourth visual metric. The video game is configured to be displayed on the device. The method further including determining a test frequency, wherein one or more of the optotype or the Amsler grid is caused to be displayed on the device based on the test frequency. The guessing rate may be based on wrong answers and unknown answers corresponding to a set of previously displayed optotypes. The method may further include calculating the distance between the user and the device based on a distance between two landmarks identified on an image of the user generated by the device. The machine learning model may include at least one convolutional neural network (CNN) and the second visual metric comprises a likelihood of a presence of one or more scotoma and/or one or more metamorphopsia.

Yet another system is provided for detecting a change in vision of a user. The system may include memory configured to store computer-executable instructions, and at least one computer processor configured to access memory and execute the computer-executable instructions to cause an optotype to be displayed on a device of the user, the optotype oriented in a first direction, receive acuity results corresponding to the optotype and first user input indicative a second direction associated with the optotype, generate a first visual metric corresponding to the user based on the first direction, the acuity results, and one or more of a distance between the user and the device or a guessing rate, cause an Amsler grid to be displayed on the device of the user, receive first Amsler results including second user input corresponding to one or more grid markings on the Amsler grid indicative of vision distortions of the user, train a machine learning model to generate at least one visual metric corresponding to Amsler results, generate a second visual metric of the user based on the first Amsler results and using the machine learning model, cause a video game to be displayed on the device, and generate a third visual metric corresponding to the user based on a user's performance in the video game.

The at least one computer processor may be further configured to access memory and execute the computer-executable instructions to generate video game metrics corresponding to the video game based on the first visual metric and the second visual metric, the video game metrics corresponding to at least a difficulty level of the video game. The at least one computer processor may be further configured to access memory and execute the computer-executable instructions to generate a fourth visual metric corresponding to the user based on the first visual metric, the second visual metric, and the third visual metric, the fourth visual metric indicative of the change in vision of the user. The at least one computer processor may further be configured to access memory and execute the computer-executable instructions to generate an alert based on one or more of the first visual metric, the second visual metric, the third visual metric, or the fourth visual metric. The at least one computer processor may be further configured to access memory and execute the computer-executable instructions to determine a test frequency, wherein one or more of the optotype or the Amsler grid is caused to be displayed on the device based on the test frequency. The machine learning model may include at least one convolutional neural network (CNN) and the second visual metric comprises a likelihood of a presence of one or more scotoma and/or one or more metamorphopsia.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 illustrates a system for testing vision, monitoring vision, and/or and estimating a change in vision.

FIG. 2A illustrates a process flow for testing vision, monitoring vision, and/or and estimating a change in vision.

FIG. 2B illustrates a psychometric curve fitted with a sigmoid function and/or logistic regression.

FIG. 3 illustrates a process flow for manipulating access to a video game based on certain performance standards.

FIG. 4 illustrates a process flow for adjusting acuity test results based on a distance to screen and/or a guessing rate.

FIG. 5 illustrates a schematic view of a trained neural network for detecting an evolution of a pathology and/or change in vision based on Amsler grids.

FIG. 6 illustrates a process flow for generating an alert based on a threshold value and the number of alerts below a threshold value.

FIG. 7 is a schematic block diagram of a computing device, in accordance with one or more example embodiments of the disclosure.

The foregoing and other features of the present technology will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

Provided herein are systems methods for testing vision of a patient, monitoring vision of a patient, determining changes in vision, and/or estimating changes in vision. The vision system may include multiple types of vision tests that a patient may access on their mobile device or other electronic device, the results of which may be collectively analyzed and used to monitor the patient's vision and/or determine and/or estimate changes in the patient's vision. For example, a patient may perform acuity tests including optotypes and may also use an Amsler grid to indicate the presence of one or more scotoma and/or metamorphopsia in the patient's field of vision. The results from the acuity tests and Amsler grids may be analyzed to determine visual metrics. The visual metrics may be used to manipulate access to and/or game play in a video game, which also may be presented on the patient's mobile device or other electronic device. The video game may serve as incentive to continue testing using the acuity tests and Amsler grids and/or may generate data indicative visual metrics of the patient based on the patient's performance in the video game. The visual metrics may be used to generate an alert corresponding to the patient's vision and/or may be used to estimate risk of evolution of a disease.

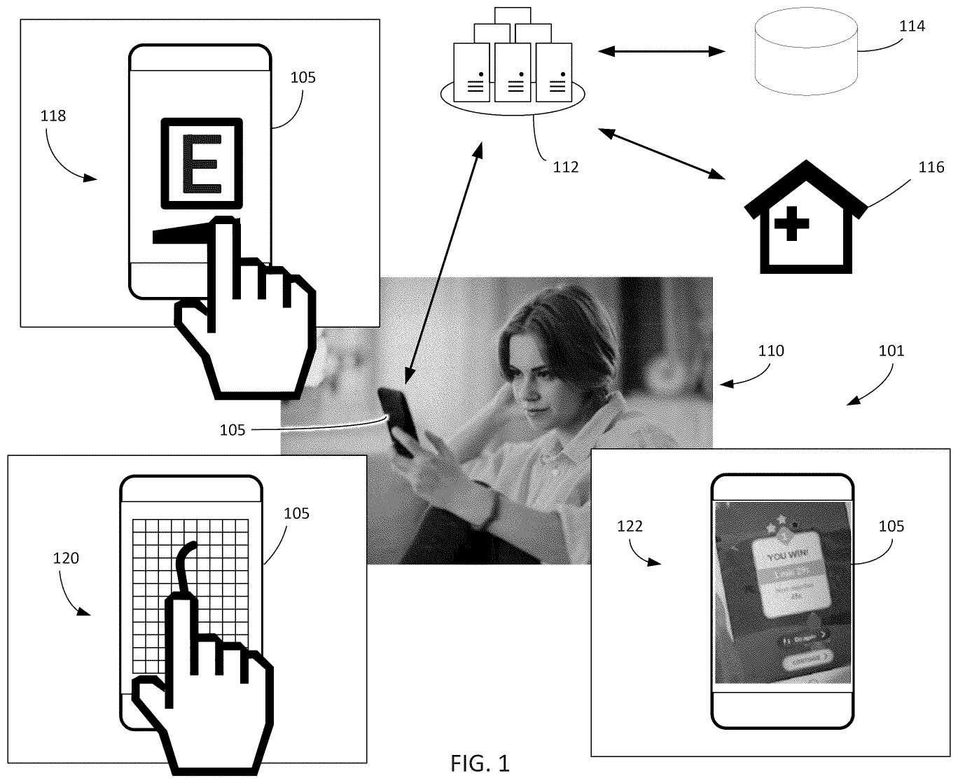

Referring now to FIG. 1 a system for testing vision of a patient, monitoring vision of a patient, determining changes in vision, and/or estimating changes in vision is depicted. As shown in FIG. 1, vision system 101 may include user device 105 which may communicate with server 112 to display vision tests and other vision information on user device 105. Server 112 may further be in communication one or more datastore 114 and/or one or more healthcare provider (HCP) device 116 as well as any other suitable computer devices.

User device 105 may be any mobile or electronic device (e.g., smart phone, tablet, laptop device, and/or any other computing devices (e.g., having memory and a processor)) for viewing vision tests (e.g., acuity tests, Amsler grids, etc.), generating user input, running one or more video games, sending and/or receiving information (e.g., alerts, emails and/or texts), accessing the internet, and the like. User device 105 may wirelessly communicate with server 112 via any suitable wireless technology (e.g., the internet, cellular, satellite, etc.).

Server 118 may be one or more, computing device (e.g., having memory and a processor), server, and/or data store. Server may further communicate with datastore 114 which may be one or more datastores (e.g., having memory and a processor) and/or may communicate with HCP device 116, which may be any smartphone, laptop, tablet, or other computing device used by a healthcare provider. Server 118 may communicate datastore 114 and/or HCP device 116 via any suitable wireless or wired technology. Datastore 114 may be used to store data and/or other information corresponding to user 110. For example, trained machine learning models unique to user 110 may be stored in datastore 114.

As shown in FIG. 1, user 110 may use device 105 to perform acuity test 118. For example, server 112 may periodically cause user device 105 to present an optotype on user device 105 that is oriented in a certain direction. User device 105 may then instruct user 110 to enter user input indicating the direction of the optotype. For example, the optotype may be facing right and user 110 may use touchscreen input on user device 105 to swipe right indicating that the optotype is oriented to the right. User device 105 may then share the user input with server 112.

As also shown in FIG. 1, server 112 may periodically cause user device 105 to present Amsler test 120, which may include presentation of an Amsler grid and instructing user 110 to provide user input indicating obstructions in the user's vision onto the Amsler grid. For example, user 110 may use the touch screen on user device 105 to indicate the size and shape of obstructions in the user's vision, which may be indicative of one or more scotoma and/or metamorphopsia. User device 105 may then share the user input corresponding to the Amsler grid with server 112 for analysis of the user input on the server. Alternatively, or additionally, the user input may be analyzed locally on user device 105.

Presenting acuity tests including optotypes as well as Amsler tests including Amsler grids are described in greater detail in U.S. Pat. No. 12,471,771 to Grondin et al., the entire contents of which are incorporated herein by reference. For example, the '771 patent provides systems and methods for testing visual acuity, an algorithm for estimating visual acuity, and systems and methods for using an Amsler grid for testing visual disturbances. It will be understood by one or ordinary skill in the art that the foregoing methods and systems for testing and estimating visual acuity and visual disturbances using an Amsler grid may be used by vision system 101.

The input data received by server 112 relating to acuity test 118 and Amsler test 120 may be used to monitor the vision of user 110 and/or detect and/or estimate changes in vision. If changes in vision are detected alerts may be generated and communicated to user 110 via user device 105 and/or HCP device 116, which may be a device used by a HCP of user 110.

The input data received by server 112 relating to acuity test 118 and Amsler test 120 may also be used to manipulate access user 110 has to video game 122, which also may be presented on user device 105. Video game 122 may be used as incentive for a user to continue to perform acuity tests 118 and/or Amsler test 120 by only providing access to video game 122 after acuity test 118 and/or Amsler test 120 are performed. Alternatively, or additionally, video game 122 may optionally be used to monitor a user's vision and/or detect changes in vision. For example, a user's performance in video game 122 may be shared with server 122 and may be processed by server 112. In one example, a poor performance by a user (e.g., in ability to beat a level or achieve a goal the user was previously able to beat or achieve) may be indicative of a change of vision. In one example, vision system 101 may use the same systems and methods for monitoring and/or testing a user's vision and/or using a user's performance in a video game to infer and/or detect changes in a user's vision described in the '771 patent. It will be understood by one skilled in the art that use of video game 122 to monitor a user's vision and/or detect changes in the user's vision may be optional in vision system 101.

The user input generated by acuity test 118, Amsler test 120, and video game 122 may collectively be processed by server 112, or alternatively, locally on user device 105, to generate visual metrics which may be an overall assessment of the user's vision, to determine changes in the user's vision, estimate a change in the user's vision, track progression of a visual disease, and/or estimate a risk of evolution of a visual disease (e.g., macular diseases, Age-related macular degeneration (AMD), cataracts, glaucoma, retinitis pigmentosa, diabetic retinopathy, etc.).

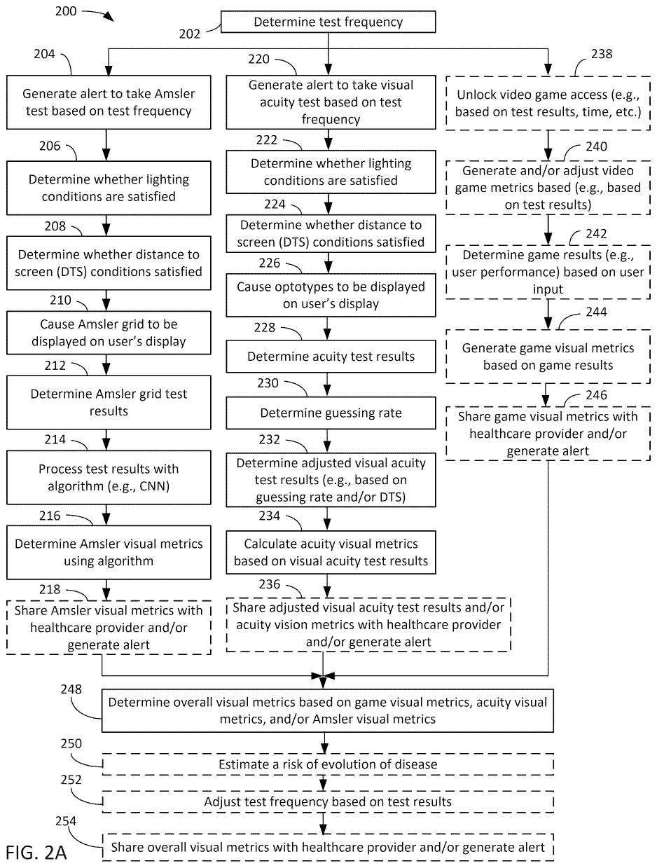

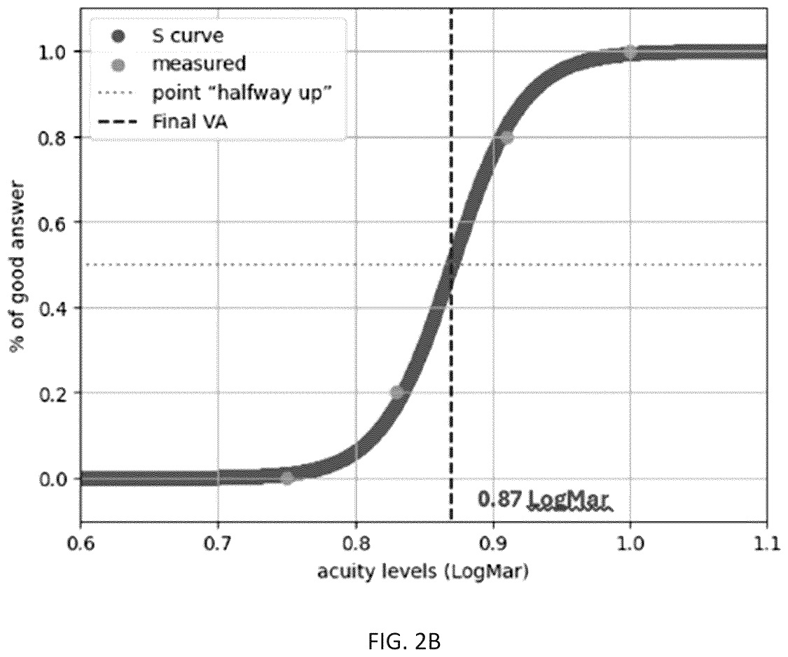

Referring now to FIG. 2A, a process flow for testing vision, monitoring vision, and/or detecting a change in vision is illustrated. Process flow 200 may be used by the vision system (e.g., vision system 101 of FIG. 1) to monitor vision, test vision, determine changes in vision, estimate an evolution of an eye disease, and/or generate alerts send to the user and/or a healthcare provider relating to the foregoing. Some or all of the blocks of the process flow in this disclosure may be performed in a distributed manner across any number of devices (e.g., a server such as server 112 and/or user device such as user device 105 of FIG. 1). Some or all of the operations of the process flow may be optional and may be performed in a different order. It will be understood by one skilled in the art that use of the video game at blocks 238-246 to monitor a user's vision and/or detect changes in the user's vision may be optional in the vision system.

To initiate process flow 200, at block 202, computer-executable instructions stored on a memory of a device, such as a server and/or user device, may be executed to determine a testing frequency. For example, a test frequency for acuity tests, Amsler tests and/or video game testing may be set by the server and user device. The frequency of testing may be the same or different for reach type of test (e.g., acuity test, Amsler test, and/or video game test). In one example, the test frequency may be every day, every other day, twice a week, once a week, every two weeks, or any other suitable frequency.

At block 204, computer-executable instructions stored on a memory of a device, such as a server and/or user device, may be executed to generate an alert (e.g., prompt on the user device) to take an Amsler test based on the test frequency. At block 206, computer-executable instructions stored on a memory of a device, such as a server and/or user device, may be executed to determine whether lighting conditions are sufficient for performing the Amsler test. For example, the ambient environment in which the user device and user are in must be sufficiently bright to perform the Amsler test. For example, the systems and methods in the '771 patent for determining whether the screen brightness and the ambient light are within a required range may used. As explained in the '771 patent, the brightness of the display of the user device may be required to be in the range from about 100 lux to about 300 lux and the ambient light may be required to be in the range from 10 lux to about 2000 lux. A light sensor (e.g., camera of the user device) may be used to determine the ambient light.

Once it is determined at block 206 that the light conditions are satisfied, at block 208, computer-executable instructions stored on a memory of a device, such as a server and/or user device, may be executed to determine whether the distance to screen (DTS) between the user's face and the screen of the user device are satisfied. For example, the systems and methods in the '771 patent for determining whether the DTS between the user's eyes and the screen may used. As explained in the '771 patent, the camera of the user device may be used to estimate the DTS based on facial landmarks (e.g., eyes, nose, mouth, etc.) and the DTS may be required to be in the range between 30 cm and 50 cm.

At block 210, computer-executable instructions stored on a memory of a device, such as a server and/or user device, may be executed to determine Amsler test results. The Amsler test may be a digitalized version of the Amsler grid (M. Amsler, Brit. J. Ophthal., Vol. 37, 521, 1953), which is a diagnostic tool that aids in the detection of visual disturbances caused by changes in the retina, particularly the macula. The Amsler grid test results may include user input generating according to systems and methods explained in the '771 patent for generating Amsler grid test results. In one example, the user may be prompted to draw lines if distortions are seen or to fill or circle area where the grid disappears while fixing a central dot. The user input may be indicative of one or more scotoma and/or metamorphopsia.

At block 212, computer-executable instructions stored on a memory of a device, such as a server and/or user device, may be executed to determine Amsler grid test results which may be the user input generated during the Amsler test. The Amsler Grid test does not give a result or a value, but instead generates annotated image of what the user has drawn (e.g., lines and/or circles).

At block 214, computer-executable instructions stored on a memory of a device, such as a server and/or user device, may be executed to process the Amsler test results with a machine learning algorithm trained to process the test results and determine the likelihood of a presence of one or more scotoma and/or metamorphopsia, the size and/or position of one or more scotoma and/or metamorphopsia, and/or an amount of scotoma and/or metamorphopsia. The trained algorithm may additionally, or alternatively, determine a change in the user's vision based on the user input corresponding to multiple Amsler tests. For example, the results generated at block 212 may be compared to previously generated results and may determine if the user's vision has changed based on changes in the user input. Optionally, at block 214, a measurement and/or level of the quality of vision may be determined. For example, each annotated Amsler grid maybe processed by a trained neural network to output a level of quality of vision based on the annotated Amsler grid input into the trained neural network.

At block 216, computer-executable instructions stored on a memory of a device, such as a server and/or user device, may be executed to determine Amsler visual metrics (e.g., using one or more algorithms, thresholds, and/or ranges). For example, the output of the trained algorithm (e.g., CNN) at block 214 may indicate a visual metric such as a likelihood of a presence of one or more scotoma and/or one or more metamorphopsia, amount data corresponding to a number or amount of scotoma and/or metamorphopsia, and/or size data corresponding to the size of the scotoma and/or metamorphopsia. Other known or suitable visual metrics may be generated at block 216.

At optional block 218, computer-executable instructions stored on a memory of a device, such as a server and/or user device, may be executed to share Amsler visual metrics with one or more healthcare provide and/or generate an alert regarding the visual metrics with the user and/or the healthcare provide. The alert may indicate a change in vision was detected and/or may instruct the user to seek medical attention.

Based on the test frequency determined at block 202, at block 220, computer-executable instructions stored on a memory of a device, such as a server and/or user device, may be executed to generate an alert (e.g., prompt on the user device) to take a visual acuity test based on the test frequency. This alert may occur at the same time or a different time than the alert generated a block 204. At block 222, computer-executable instructions stored on a memory of a device, such as a server and/or user device, may be executed to determine whether lighting conditions are satisfied. At block 224, computer-executable instructions stored on a memory of a device, such as a server and/or user device, may be executed to determine whether distance to screen (DTS) conditions are satisfied. Blocks 222 and 224 may be the same as or similar to blocks 206 and 208.

At block 226, computer-executable instructions stored on a memory of a device, such as a server and/or user device, may be executed to cause one or more optotypes to be displayed on the display of the user device. A user may be prompted to indicate the direction and/or orientation of the optotype (e.g., using a touch screen display on the user device). The systems and methods for performing the acuity test and displaying the optotypes may be the same as or similar to the system and methods for the acuity test and displaying the optotypes described in the '771 patent. For example, the orientation and/or the size of the optotype may change after each patient response and the optotype orientation may be random, but its size may change according to a predefined algorithm (e.g., the size may decrease in response to correct orientation response). Alternatively, or additionally, other visual acuity tests may be presented such as the visual acuity test described in U.S. Pat. No. 12,569,131 to Grondin et al., the entire contents of which are incorporated herein by reference. For example, the acuity test may be a Vernier acuity test. Any other suitable acuity test may be presented at block 226. In one example, progressively smaller letters may be presented to the user (e.g., 1 letter per acuity level). The final level may be the starting point for the next test.

At block 228, computer-executable instructions stored on a memory of a device, such as a server and/or user device, may be executed to determine the acuity test results, which may be based on the user's input indicating the direction of the optotype. Optionally, an adjusted acuity test results may be determined based on the DTS determined. For example, based on the determined distance between the user's eyes and the screen of the user device, a more precise acuity test result may be determined. For example, for each optotype presented, a DTS value may be determined and the real acuity tested may be adjusted based on the DTS. The adjusted acuity results may be plotted against the percentage of right answers for a given adjusted acuity result (e.g., percentage of right answers given a certain size of optotype). The acuity level or size may be scored with reference to the Logarithm of the Minimum Angle of Resolution, also known as LogMAR values. A psychometric curve may be fitted for the acuity test results and adjusted results using a sigmoid function and/or logistic regression with the x-axis representing the acuity results (e.g., both raw or original value and adjusted value) and the y-axis representing the percentage of correct answers. Two curves may be fitted, one for the original or raw results and one for the adjusted results.

An exemplary psychometric curve fitted with a sigmoid function and/or logistic regression is illustrated in FIG. 2B. The psychometric curve is S-shaped. A shift may determine the curve's horizontal position and provides a measure of response bias. Scale may be inversely related to the slope of the curve and corresponds to a measure of sensitivity. Lapse rate may determine the asymptotes of the curve. As behavior during a lapse is independent of signal intensity, an error can result even if stimulus intensity is well above or below the operator's response threshold. The effect of lapses on the upper and lower asymptotes may depend on the operator's guess rate, which may be the probability of making a positive response when behavior is not stimulus driven.

Referring again to FIG. 2A, at block 230, computer-executable instructions stored on a memory of a device, such as a server and/or user device, may be executed to determine a guessing rate of the user. For example, based on the psychometric curve, a threshold of a random good answer (e.g., the guessing rate) may be estimated. Additionally, or alternatively, the acuity test display may include a button for “I don't know” or other similar button for a user to engage if the user does not know the answer. In this example, the user input may indicate the direction of the optotype and/or engagement of the “I don't know” or similar button. The guessing rate may alternatively and/or additionally be estimated by comparing the sum of “I don't know” answer and the sum of “bad answer”. In one example, if the sum of “I don't know” answers divided by the sum of “bad answers” is greater than or equal to 2, then the guessing rate may be determined to be 0. If the sum of “I don't know” answers divided by the sum of “bad answers” is less than or equal to 0.5, the guessing rate is 25%. If the sum of “I don't know” answers divided by the sum of “bad answers” is less than 2 but greater than 0.5, then the guessing rate is 12.5%. In another example, if there are more, or equal to, twice the number of bad answers than “I don't know” answers, then the guessing rate is 0; if there are more, or equal to, twice the number of “I don't know” answers than bad answers, then the guessing rate is 0.25; and if the ratio between “I don't know answers” and “bad answers” is between the previous range, then the guessing rate is 0.125. It will be understood by those skilled in the art that other suitable algorithms and/or determinations of the guessing rate may be used.

At block 232, computer-executable instructions stored on a memory of a device, such as a server and/or user device, may be executed to determine a final visual acuity value based on the guessing rate and/or the adjusted acuity value (e.g., based on the DTS). For example, the exact visual acuity value may be reduced a certain amount depending on a guessing rate greater than 0. Alternatively, or additionally, the guessing rate may be used together with the psychometric curve to determine a final visual acuity value. For example, the lower asymptote and/or lapse rate may correspond to the guessing rate and an exact acuity value may be determined based on the psychometric curve.

In one example, the acuity value may be adjusted based on the DTS value. For example, If the ideal (e.g., desired and/or theoretical) DTS is 40 cm to achieve the desired acuity size, the real DTS may be calculated and the results adjusted to achieve a DTS of 40 cm. To adjust the acuity results based on the DTS, an average DTS may be determined for multiple measurements (e.g., during multiple tests or multiple measurements determined during a single test).

Alternatively, one a single measurement may be used. The difference between the average DTS and a desired distance to screen (e.g., 40 cm) may be determined. For example, the difference may be the difference between the desired DTS and the average DTS. If the average DTS is 38 cm and the desired DTS is 40 cm then the difference (i.e., delta) is “2 cm.” To convert the new visual acuity into LOGMAR units, in one example the following formula is used:

$$Delta\_LogMar = Log [ 1 + ( \frac{Delta}{desired DTS} ) ]$$

The result may be rounded (e.g., to the nearest 100^{th} value). In one example, the relationship between a letter and LogMar units is +1 letter is equivalent to −0.02 LogMar (i.e., −0.02 LogMar=1 letter). For example, the following values may be used for determining the adjusted letter value based on the rounded score: Delta_LogMar 0.01=Delta letter 0.5, Delta_LogMar 0.02=Delta letter 1; Delta LogMar 0.03=Delta letter 1.5; Delta_LogMar 0.04=Delta letter 2; Delta_LogMar 0.05=Delta letter 2.5; Delta_LogMar 0.06=Delta letter 3; Delta_LogMar 0.07=Delta letter 3.5; Delta_LogMar 0.08=Delta letter 4; Delta_LogMar 0.09=Delta letter 4.5; and Delta_LogMar 0.1=Delta letter 5. The letter value (e.g., acuity on letter) corresponding to the desired DTS (e.g., 40 cm and acuity on letter value of 50) may be adjusted based on the delta LogMar value calculated. For example, if the Delta LogMar value is 0.02 corresponding to −1 delta letter and the acuity on letter value corresponding to the desired DTS is 50, then the new visual acuity value (e.g., the new letter value) is 49 (e.g., subtracted because the difference between the actual DTS and the desired DTS is negative).

At block 234, computer-executable instructions stored on a memory of a device, such as a server and/or user device, may be executed to determine and/or calculate acuity visual metrics based on the visual acuity test results, adjusted results based on DTS, and/or exact results based on the guessing rate. For example, the visual metrics may be a value indicative of a change in vision, of an evolution of an eye and/or vision disease, and/or any suitable visual metric.

At block 236, computer-executable instructions stored on a memory of a device, such as a server and/or user device, may be executed to share acuity visual metrics based with one or more healthcare provider and/or generate an alert regarding the visual metrics with the user and/or the healthcare provider. The alert may indicate a change in vision was detected and/or may instruct the user to seek medical attention, for example.

Additionally, after determining test frequency at block 202, and/or after determining Amsler grid test results at block 212, Amsler visual metrics at block 216, adjusted visual acuity test results at block 232, and/or acuity visual metrics at block 234, optional block 238 may be initiated. At optional block 238, computer-executable instructions stored on a memory of a device, such as a server and/or user device, may be executed to unlock video game access on the user device. For example, access to the video game generally and/or access to certain levels and/or functionality may be unlocked or otherwise manipulated. The video game may be a puzzle based game, a maze type game, an adventure game, a role play game, and/or any other suitable video game. The generation of visual acuity results and/or Amsler grid test results may indicate that the user is in compliance with the prescribed testing frequency, which may be sufficient to unlock user access to the video game and/or to the next level in a video game or other game play.

At optional block 240, computer-executable instructions stored on a memory of a device, such as a server and/or user device, may be executed to generate and/or adjust video game metrics based on Amsler visual metrics at block 216, adjusted visual acuity test results at block 232, and/or acuity visual metrics at block 234, for example. The video game metrics that may be adjusted may include a difficulty level, access to an advanced level, and/or access to certain functionality, for example.

At optional block 242, computer-executable instructions stored on a memory of a device, such as a server and/or user device, may be executed to determine game results (e.g., user performance in the video game) based on the user input. For example, a number of points, goals achieved, levels passed, time elapsed, or any other suitable video game metric may be considered when considering user performance. At block 244, computer-executable instructions stored on a memory of a device, such as a server and/or user device, may be executed to generate game visual metrics based on game results. For example, game visual metrics may indicate a change in vision if the user was once able to achieve a certain performance metric (e.g., level, time elapsed, goal, etc.) and is no longer achieving the same metric, for example. An algorithm and/or trained neural network may process the user's input in the video game to determine the game visual metrics.

At optional block 246, computer-executable instructions stored on a memory of a device, such as a server and/or user device, may be executed to share game visual metrics with one or more healthcare provider and/or generate an alert regarding the visual metrics with the user and/or the healthcare provider. The alert may indicate a change in vision was detected and/or may instruct the user to seek medical attention, for example.

At block 248, computer-executable instructions stored on a memory of a device, such as a server and/or user device, may be executed to determine overall visual metrics based on acuity visual metrics, Amsler visual metrics, and/or game visual metrics. For example, each of acuity visual metrics, Amsler visual metrics, and/or game visual metrics may be weighted and combined, may be averaged, and/or may be combined in any other suitable fashion to determine over visual metrics for the user, indicative the state of a user's vision, a change of a the user's vision, or the like. Analyzing the combination of acuity visual metrics and Amsler visual metrics, and also optionally game visual metrics, facilitates more accurate estimation of risk of evolution of the disease as each of the Amsler grid test, visual acuity test, and the video game test present different visual challenges and test different eye functionality and visual performance. Considering the corresponding metrics and results together facilitate a more accurate estimate of risk of the evolution of the disease than the current systems which follow a more siloed approach.

At optional block 250, computer-executable instructions stored on a memory of a device, such as a server and/or user device, may be executed to determine an estimate of a risk of evolution and/or progression of an eye disease. For example, the overall visual metric at block 248, the Amsler visual metric at block 216, the acuity visual metric at block 234, and/or the game visual metric at block 244 may be used to determine evolution and/or progression of an eye disease (e.g., an algorithm may process the data and generate an output indicative of evolution and/or progression of an eye disease).

At optional block 252, computer-executable instructions stored on a memory of a device, such as a server and/or user device, may be executed to adjust the test frequency for the Amsler grid test, acuity test, and/or video game testing based on the overall visual metric at block 248, the Amsler visual metric at block 216, the acuity visual metric at block 234, and/or the game visual metric at block 244 and/or any other test results relating to the Amsler grid test, the visual acuity test, and/or the video game test. For example, if a loss of vision is detected, the test frequency may be increased (e.g., from every other day to every day).

At optional block 254, computer-executable instructions stored on a memory of a device, such as a server and/or user device, may be executed to share the overall visual metrics with one or more healthcare provider and/or the user. Alternatively, or additionally, an alert may be generated and sent to the healthcare provider and/or user indicating a change of vision indicated by the overall visual metric.

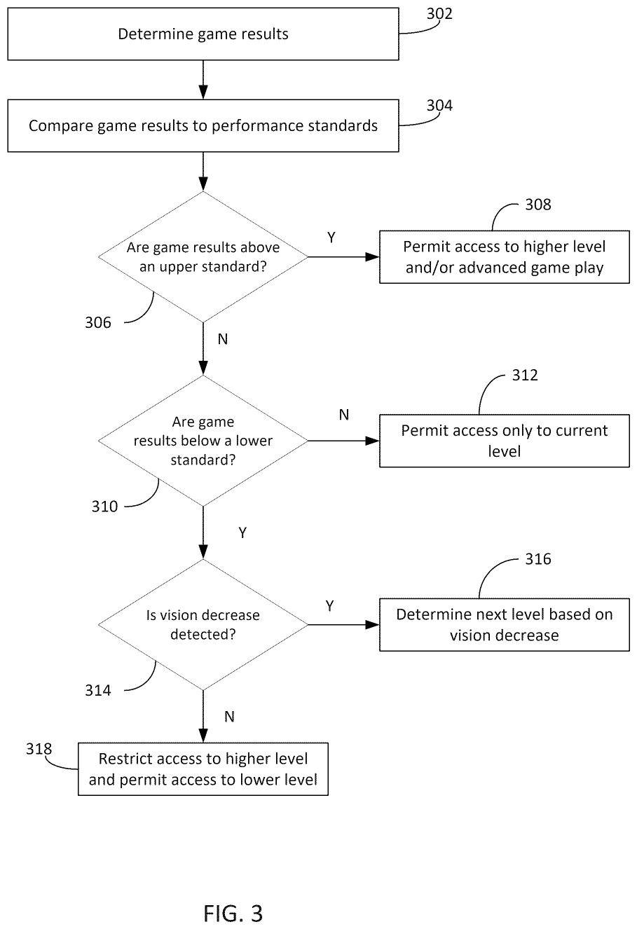

Referring now to FIG. 3, a process flow for manipulating access to a video game based on certain performance standards is illustrated. Some or all of the blocks of the process flow in this disclosure may be performed in a distributed manner across any number of devices (e.g., a server such as server 112 and/or user device such as user device 105 of FIG. 1). Some or all of the operations of the process flow may be optional and may be performed in a different order.

To initiate the process flow in FIG. 3, at block 302, computer-executable instructions stored on a memory of a device, such as a server and/or user device, may be executed to determine game results based on user performance. At block 304, computer-executable instructions stored on a memory of a device, such as a server and/or user device, may be executed to compare the game results to certain performance standards. For example, metrics such as levels passed, time elapsed, goals achieved, and the like may be included in game results and the performance standards may include certain number of game levels, a certain time elapsed, a type and/or number of goals achieved, and the like.

At decision 306, computer-executable instructions stored on a memory of a device, such as a server and/or user device, may be executed to determine whether the game results are above an upper level of performance standards. If the game results are above an upper level performance standard (e.g., a certain level was passed in an certain amount of time), at block 308 computer-executable instructions stored on a memory of a device, such as a server and/or user device, may be executed to permit access to a higher level and/or advanced game play. For example, a more advanced level of game play, advanced functionality, and/or other advanced game play may be unlocked.

Alternatively, if the game results are not above an upper level performance standard, at decision 310, computer-executable instructions stored on a memory of a device, such as a server and/or user device, may be executed to determine whether the game results are below a certain lower level performance standard (e.g., a level was not passed or was passed after a set amount of time). If the game results are not below a lower level performance standard, at block 312 computer-executable instructions stored on a memory of a device, such as a server and/or user device, may be executed to permit access only to the current level and/or current level of play and/or game functionality.

Alternatively, if the game results are not above an upper performance standard and are below a lower level performance standard, at decision 314 computer-executable instructions stored on a memory of a device, such as a server and/or user device, may be executed to determine if a vision decrease is detected. If a vision decrease is detected, at block 316, computer-executable instructions stored on a memory of a device, such as a server and/or user device, may be executed to adjust the level of game play and/or the functionality available in the video game based on the vision decrease detected. Alternatively, if a vision decrease is not detected, at block 318 computer-executable instructions stored on a memory of a device, such as a server and/or user device, may be executed to restrict access to a higher level of the video game or advanced game play and permit access to a lower level of the video game. Additionally, certain functionality and/or game play may be restricted or removed.

Gating game play in this manner facilitates testing of visual metrics and/or identifying decreases in vision metrics while the user benefits from the enjoyment and challenges of a video game. The challenges presented by the video game and game advancement (e.g., achieving a goal or passing a level) incentivize individuals to play the game. The incorporation of the video game play into a visual testing system is an improvement over current visual testing systems as it encourages compliance by the individual, can be used for testing visual metrics, and may also provide joy and entertainment to the individual.

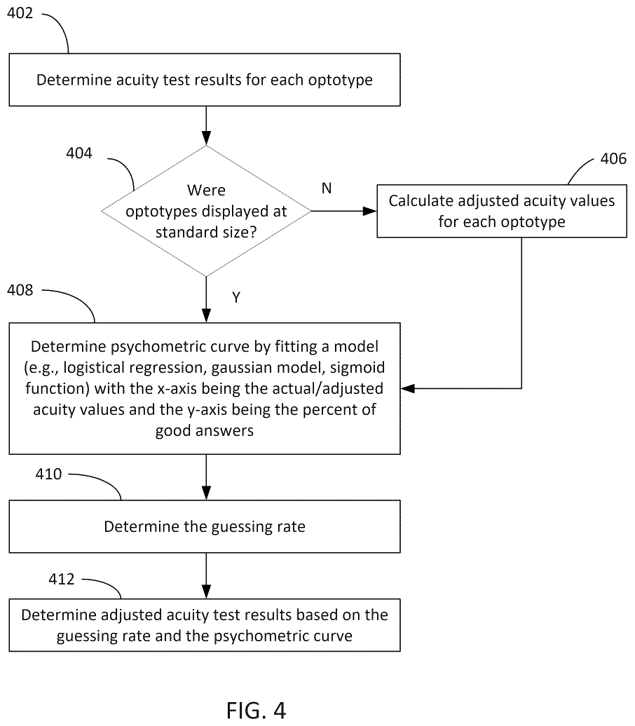

Referring now to FIG. 4, a process flow for adjusting acuity test results based on a guessing rate and/or psychometric curve is illustrated. Some or all of the blocks of the process flow in this disclosure may be performed in a distributed manner across any number of devices (e.g., a server such as server 112 and/or user device such as user device 105 of FIG. 1). Some or all of the operations of the process flow may be optional and may be performed in a different order.

The process illustrated in FIG. 4 may be initiated at block 402 at which computer-executable instructions stored on a memory of a device, such as a server and/or user device, may be executed to determine acuity test results (e.g., user input received in response to an acuity test) for each optotype. At decision 404, computer-executable instructions stored on a memory of a device, such as a server and/or user device, may be executed to determine whether optotypes were displayed at standard size. This may be determined by considering the distance to the screen (DTS). If the optotypes were not displayed at the standard size, at block 406 computer-executable instructions stored on a memory of a device, such as a server and/or user device, may be executed to calculate adjusted acuity values (e.g., based on the DTS) for each optotype.

At block 408, computer-executable instructions stored on a memory of a device, such as a server and/or user device, may be executed to determine a psychometric curve by fitting a model (e.g., logistical regression, gaussian model, sigmoid function) with the x-axis being the actual/adjusted acuity values and the y-axis being the percent of good answers. At block 410, computer-executable instructions stored on a memory of a device, such as a server and/or user device, may be executed to determine the guessing rate of the user (e.g., based on the lower asymptote of the psychometric curve). At block 412, computer-executable instructions stored on a memory of a device, such as a server and/or user device, may be executed to determine adjusted acuity test results based on the guessing rate and/or the psychometric curve.

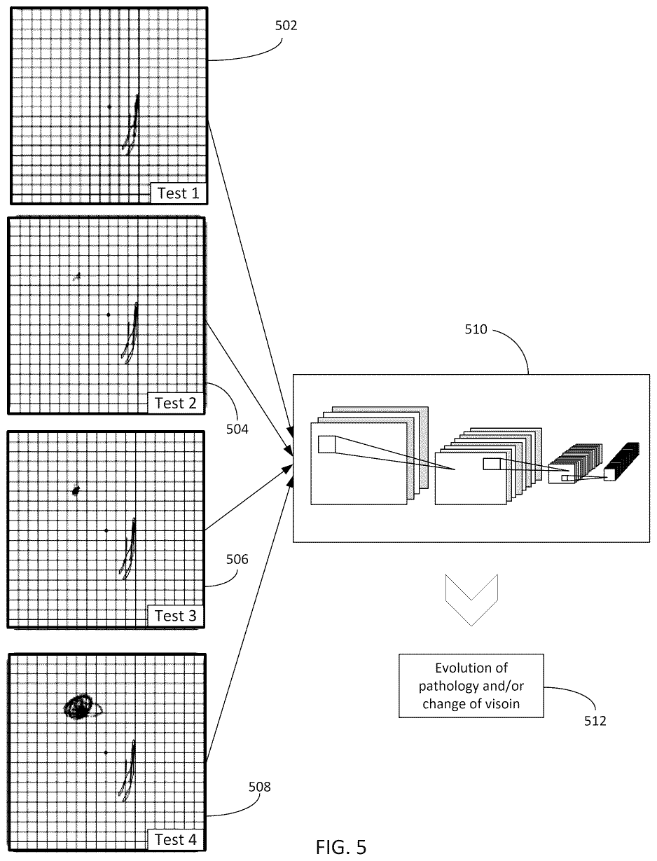

Referring now to FIG. 5, a schematic view of a trained neural network for detecting an evolution of a pathology and/or change in vision based on Amsler grids is illustrated. For example, Amsler grids 502, 504, 506, and 508 may be annotated with user input which may include lines and/or circles indicating distortion in the user's field of vision (e.g., indicating one or more scotoma and/or one or more metamorphopsia). Each of Amsler grids 502, 504, 506, and 508 may indicate different user input from different Amsler tests at different times.

Amsler grids 502, 504, 506, and 508 and/or the user input with the annotations to the grids may be processed by machine learning model 510 which may be one or more machine learning model trained to process Amsler grids. For example, a convolutional neural network (CNN) may be trained to process Amsler grids and/or user input, detect or more scotoma and/or one or more metamorphopsia and/or a size or amount of the scotoma and/or metamorphopsia. Machine learning model 510 may generate output 512 which may include values indicative of an evolution of a patient's pathology and/or a change in vision for example based on the Amsler grids and/or user input that is input into machine learning model 510.

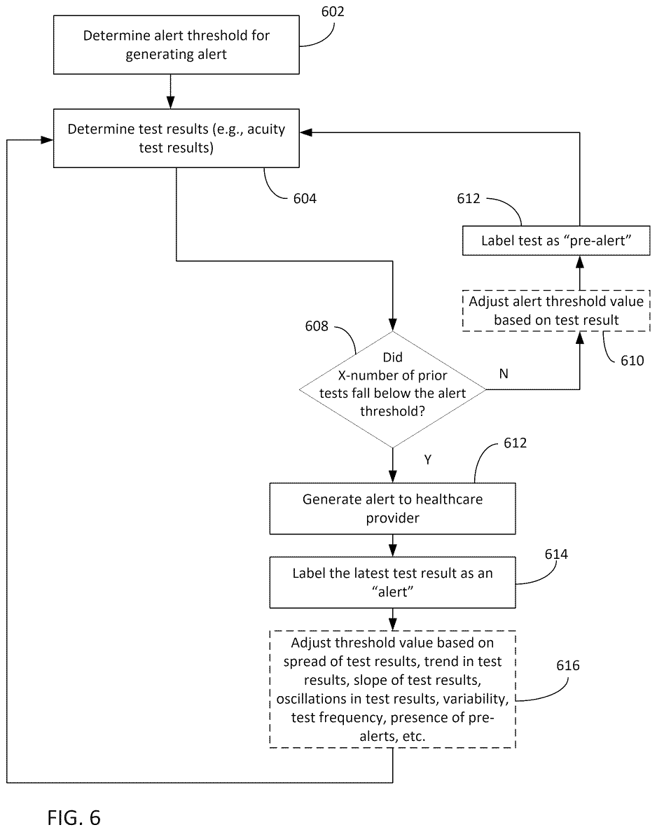

Referring now to FIG. 6, a process flow for generating an alert based on a threshold value and the number of alerts below a threshold value is illustrated. Some or all of the blocks of the process flow in this disclosure may be performed in a distributed manner across any number of devices (e.g., a server such as server 112 and/or user device such as user device 105 of FIG. 1). Some or all of the operations of the process flow may be optional and may be performed in a different order.

The process illustrated in FIG. 6 may be initiated at block 602 at which computer-executable instructions stored on a memory of a device, such as a server and/or user device, may be executed to determine an alert threshold for generating an alert. For example, the alert threshold may be a decrease in vision by a certain amount, a visual acuity test result below a certain amount, an Amsler test result below a certain amount, a video game result below a certain amount, and/or certain visual metrics below a certain amount. At block 604, computer-executable instructions stored on a memory of a device, such as a server and/or user device, may be executed to determine test results (e.g., visual acuity test results, Amsler test results, and/or video game test results).

If the test results are not below the alert threshold, block 604 may be reinitiated. If the test results are below the alert threshold or otherwise do not satisfy the alert threshold, decision 734, computer-executable instructions stored on a memory of a device, such as a server and/or user device, may be executed to determine whether a certain number of prior test tests fall below the alert threshold (e.g., at least one immediately prior test result). If the certain number of prior tests did not fall below the threshold, at optional block 610 computer-executable instructions stored on a memory of a device, such as a server and/or user device, may be executed to adjust the alert threshold value based on the test result (e.g., the value at block 602 may be raised or lowered). At block 612, computer-executable instructions stored on a memory of a device, such as a server and/or user device, may be executed to label the test results with a “pre-alert” label.

Alternatively, if the certain number of prior tests did fall below the alert threshold or otherwise did not satisfy the alert threshold (e.g., the immediately prior test also fell below the alert threshold), at block 612 computer-executable instructions stored on a memory of a device, such as a server and/or user device, may be executed to generate an alert to a healthcare provider and optionally the user device, indicating that one or more test results fell below a threshold value or otherwise did not satisfy a threshold value.

At block 614, computer-executable instructions stored on a memory of a device, such as a server and/or user device, may be executed to label the test result generated at block 604 as an “alert” indicating that an alert was generated for this test result. At optional block 616, computer-executable instructions stored on a memory of a device, such as a server and/or user device, may be executed to adjust the threshold value at block 602. For example, the threshold value may be adjusted to be more or less sensitive based on the spread of test results, trend in test results, slope of the test results, oscillations in test results, variability in test frequency, and/or presence of pre-alerts, and the like.

In one example, the threshold may be determined by a low percentile corresponding to the 25th percentile of previous visual acuity values observed, a high percentile, corresponding to the 75th percentile of previous visual acuity values, a coefficient (e.g., weight) to weight the dispersion of the reference values. A higher coefficient may mean greater variability, and a lower coefficient may mean less variability. The coefficient may be greater than 1 and depend on the frequency with which patients are connected. The number of letters allowed may be depend on the connection frequency and the oscillations of the visual acuity test curve. For example, a first threshold may be equal to the high percentile subtracted by the difference between high percentile and the low percentile multiplied by the weight. A second threshold may be equal to the high percentile minus the number of letters.

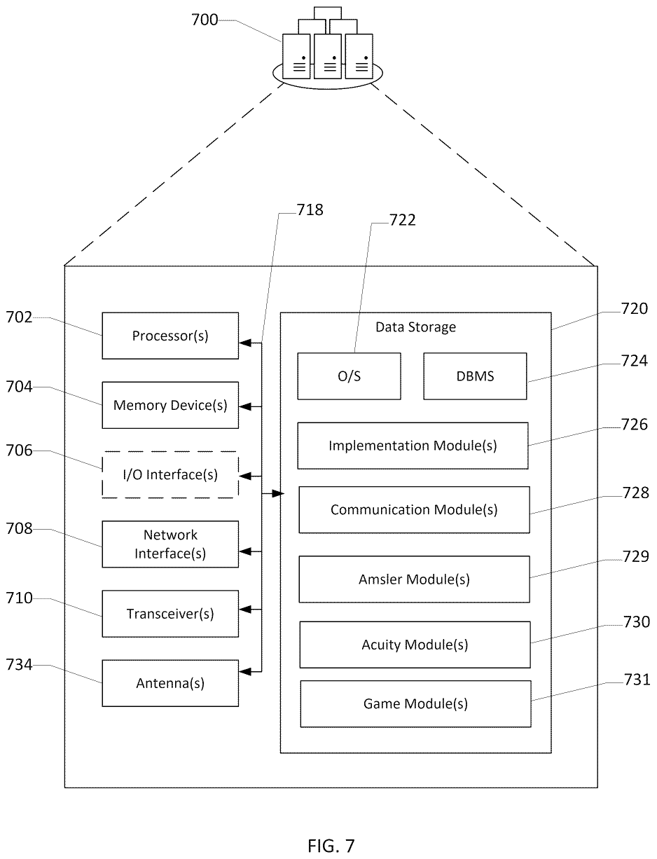

Referring now to FIG. 7, a schematic block diagram of server 700 is illustrated. Server 700 may be the same or similar to server 112 of FIG. 1 or otherwise one or more of the servers of FIGS. 1-6. It is understood that server 700 may alone or together with any user device (e.g., user device 105 of FIG. 1) perform one or more of the operations of server 700 described herein.

Server 700 may be designed to communicate with one or more servers, user devices, healthcare provider devices, data stores, other systems, or the like. Server 700 may be designed to communicate via one or more networks. Such network(s) may include, but are not limited to, any one or more different types of communications networks such as, for example, cable networks, public networks (e.g., the Internet), private networks (e.g., frame-relay networks), wireless networks, cellular networks, telephone networks (e.g., a public switched telephone network), or any other suitable private or public packet-switched or circuit-switched networks.

In an illustrative configuration, server 700 may include one or more processors 702, one or more memory devices 704 (also referred to herein as memory 704), one or more input/output (I/O) interface(s) 706, one or more network interface(s) 708, one or more transceiver(s) 710, one or more antenna(s) 734, and data storage 720. The server 700 may further include one or more bus(es) 718 that functionally couple various components of the server 700.

The bus(es) 718 may include at least one of a system bus, a memory bus, an address bus, or a message bus, and may permit exchange of information (e.g., data (including computer-executable code), signaling, etc.) between various components of the server 700. The bus(es) 718 may include, without limitation, a memory bus or a memory controller, a peripheral bus, an accelerated graphics port, and so forth. The bus(es) 718 may be associated with any suitable bus architecture including.

The memory 704 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In various implementations, the memory 704 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth.

The data storage 720 may include removable storage and/or non-removable storage including, but not limited to, magnetic storage, optical disk storage, and/or tape storage. The data storage 720 may provide non-volatile storage of computer-executable instructions and other data. The memory 704 and the data storage 720, removable and/or non-removable, are examples of computer-readable storage media (CRSM) as that term is used herein. The data storage 720 may store computer-executable code, instructions, or the like that may be loadable into the memory 704 and executable by the processor(s) 702 to cause the processor(s) 702 to perform or initiate various operations. The data storage 720 may additionally store data that may be copied to memory 704 for use by the processor(s) 702 during the execution of the computer-executable instructions. Moreover, output data generated as a result of execution of the computer-executable instructions by the processor(s) 702 may be stored initially in memory 704, and may ultimately be copied to data storage 720 for non-volatile storage.

The data storage 720 may store one or more operating systems (O/S) 722; one or more optional database management systems (DBMS) 724; and one or more program module(s), applications, engines, computer-executable code, scripts, or the like such as, for example, one or more implementation modules 726, communication modules 728, Amsler modules 729, acuity modules 730, and/or game modules 731. Some or all of these modules may be sub-modules. Any of the components depicted as being stored in data storage 720 may include any combination of software, firmware, and/or hardware. The software and/or firmware may include computer-executable code, instructions, or the like that may be loaded into the memory 704 for execution by one or more of the processor(s) 702. Any of the components depicted as being stored in data storage 720 may support functionality described in reference to correspondingly named components earlier in this disclosure.

Referring now to other illustrative components depicted as being stored in the data storage 720, the O/S 722 may be loaded from the data storage 720 into the memory 704 and may provide an interface between other application software executing on the server 700 and hardware resources of the server 700. More specifically, the O/S 722 may include a set of computer-executable instructions for managing hardware resources of the server 700 and for providing common services to other application programs (e.g., managing memory allocation among various application programs). In certain example embodiments, the O/S 722 may control execution of the other program module(s) to for content rendering. The O/S 722 may include any operating system now known or which may be developed in the future including, but not limited to, any server operating system, any mainframe operating system, or any other proprietary or non-proprietary operating system.

The optional DBMS 724 may be loaded into the memory 704 and may support functionality for accessing, retrieving, storing, and/or manipulating data stored in the memory 704 and/or data stored in the data storage 720. The DBMS 724 may use any of a variety of database models (e.g., relational model, object model, etc.) and may support any of a variety of query languages. The DBMS 724 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like.

The optional input/output (I/O) interface(s) 706 may facilitate the receipt of input information by the server 700 from one or more I/O devices as well as the output of information from the server 700 to the one or more I/O devices. The I/O devices may include any of a variety of components such as a display or display screen having a touch surface or touchscreen; an audio output device for producing sound, such as a speaker; an audio capture device, such as a microphone; an image and/or video capture device, such as a camera; and so forth. Any of these components may be integrated into the server 700 or may be separate.

The server 700 may further include one or more network interface(s) 708 via which the server 700 may communicate with any of a variety of other systems, platforms, networks, devices, and so forth. The network interface(s) 708 may enable communication, for example, with one or more wireless routers, one or more host servers, one or more web servers, and the like via one or more of networks.

The antenna(s) 734 may include any suitable type of antenna depending, for example, on the communications protocols used to transmit or receive signals via the antenna(s) 734. Non-limiting examples of suitable antennas may include directional antennas, non-directional antennas, dipole antennas, folded dipole antennas, patch antennas, multiple-input multiple-output (MIMO) antennas, or the like. The antenna(s) 734 may be communicatively coupled to one or more transceivers 712 or radio components to which or from which signals may be transmitted or received. Antenna(s) 734 may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals including BLE signals, a Near Field Communication (NFC) antenna for transmitting or receiving NFC signals, a 900 MHz antenna, and so forth.

The transceiver(s) 712 may include any suitable radio component(s) for, in cooperation with the antenna(s) 734, transmitting or receiving radio frequency (RF) signals in the bandwidth and/or channels corresponding to the communications protocols utilized by the server 700 to communicate with other devices. The transceiver(s) 712 may include hardware, software, and/or firmware for modulating, transmitting, or receiving-potentially in cooperation with any of antenna(s) 734-communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. The transceiver(s) 612 may further include hardware, firmware, or software for receiving GNSS signals. The transceiver(s) 712 may include any known receiver and baseband suitable for communicating via the communications protocols utilized by the server 700. The transceiver(s) 612 may further include a low noise amplifier (LNA), additional signal amplifiers, an analog-to-digital (A/D) converter, one or more buffers, a digital baseband, or the like.

Referring now to functionality supported by the various program module(s) depicted in FIG. 7, implementation module(s) 726 may include computer-executable instructions, code, or the like that responsive to execution by one or more of the processor(s) 702 may perform functions including, but not limited to, overseeing coordination and interaction between one or more modules and computer executable instructions in data storage 720, overseeing execution of one or more modules in the vision system, determining user input, and sharing input, and/or other vision information with other devices in the vision system.

Communication module(s) 728 may include computer-executable instructions, code, or the like that responsive to execution by one or more of the processor(s) 702 may perform functions including, but not limited to, communicating with one or more devices, for example, via wired or wireless communication, communicating with servers (e.g., remote servers), communicating with datastores and/or databases, communicating with user devices and/or user devices, sending or receiving notifications or commands/directives, communicating with cache memory data, communicating with computing devices, and the like.

The Amsler module(s) 729 may include computer-executable instructions, code, or the like that responsive to execution by one or more of the processor(s) 702 may perform functions including, but not limited to, overseeing Amsler testing, receiving user input corresponding to the Amsler test, processing the user input, and/or generating visual metrics and other information relating to Amsler testing.

Acuity module(s) 730 may include computer-executable instructions, code, or the like that responsive to execution by one or more of the processor(s) 702 may perform functions including, but not limited to, overseeing acuity testing, receiving user input corresponding to the acuity testing, processing the user input, and/or generating visual metrics and other information relating to acuity testing.

Game module(s) 731 may include computer-executable instructions, code, or the like that responsive to execution by one or more of the processor(s) 702 may perform functions including, but not limited to, overseeing video game testing, receiving user input corresponding to the video game, processing the user input, and/or generating visual metrics and other information relating to the video game.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, or the like disclosed herein may include one or more software components, including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component including assembly language instructions may require conversion into executable machine code by an assembler prior to execution by the hardware architecture and/or platform.

Another example programming language may be a higher-level programming language that may be portable across multiple architectures. A software component including higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component including instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may include other custom-developed application software, operating system functionality (e.g., device drivers, data storage (e.g., file management) routines, other common routines, and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for detecting a change in vision of a user, the method comprising:
causing an optotype to be displayed on a device of the user, the optotype oriented in a first direction;
receiving acuity results corresponding to the optotype and first user input indicative a second direction associated with the optotype;
generating a first visual metric corresponding to the user based on the first direction, the acuity results, and one or more of a distance between the user and the device or a guessing rate;
causing an Amsler grid to be displayed on the device;
receiving first Amsler results comprising second user input corresponding to one or more grid markings on the Amsler grid indicative of vision distortions of the user;
training a machine learning model to generate at least one visual metric corresponding to Amsler results;
generating a second visual metric of the user based on the Amsler results and using the machine learning model; and
generating video game metrics corresponding to a video game based on the first visual metric and the second visual metric, the video game metrics corresponding to at least a difficulty level of the video game.

2. The method of claim 1, further comprising generating a third visual metric based on at least the first visual metric and the second visual metric, the third visual metric indicative of the change in vision of the user.

3. The method of claim 1, further comprising generating a third visual metric corresponding to the user based on a user's performance in the video game.

4. The method of claim 3, further comprising generating a fourth visual metric corresponding to the user based on the first visual metric, the second visual metric, and the third visual metric, the fourth visual metric indicative of the change in vision of the user.

5. The method of claim 4, further comprising generating an alert based on one or more of the first visual metric, the second visual metric, the third visual metric, or the fourth visual metric.

6. The method of claim 1, wherein the video game is configured to be displayed on the device.

7. The method of claim 1, further comprising determining a test frequency, wherein one or more of the optotype or the Amsler grid is caused to be displayed on the device based on the test frequency.

8. The method of claim 1, wherein the guessing rate is based on wrong answers and unknown answers corresponding to a set of previously displayed optotypes.

9. The method of claim 1, further comprising calculating the distance between the user and the device based on a distance between two landmarks identified on an image of the user generated by the device.

10. The method of claim 1, wherein the machine learning model comprises at least one convolutional neural network (CNN) and the second visual metric comprises one or more of a likelihood of a presence of one or more scotoma and/or one or more metamorphopsia, amount data corresponding to the one or more scotoma and/or the one or more metamorphopsia, and/or size data corresponding to the one or more scotoma and/or the one or more metamorphopsia.

11. A system for detecting a change in vision of a user, the system comprising:
memory configured to store computer-executable instructions; and
at least one computer processor configured to access memory and execute the computer-executable instructions to:
cause an optotype to be displayed on a device of the user, the optotype oriented in a first direction;
receive acuity results corresponding to the optotype and first user input indicative a second direction associated with the optotype;
generate a first visual metric corresponding to the user based on the first direction, the acuity results, and one or more of a distance between the user and the device or a guessing rate;
cause an Amsler grid to be displayed on the device;
receive first Amsler results comprising second user input corresponding to one or more grid markings on the Amsler grid indicative of vision distortions of the user;
train a machine learning model to generate at least one visual metric corresponding to Amsler results;
generate a second visual metric of the user based on the Amsler results and using the machine learning model; and
generate video game metrics corresponding to a video game based on the first visual metric and the second visual metric, the video game metrics corresponding to at least a difficulty level of the video game.

12. The system of claim 1, wherein the at least one computer processor is further configured to access memory and execute the computer-executable instructions to generate a third visual metric corresponding to the user based on a user's performance in the video game.

13. The system of claim 12, wherein the at least one computer processor is further configured to access memory and execute the computer-executable instructions to generate a fourth visual metric corresponding to the user based on the first visual metric, the second visual metric, and the third visual metric, the fourth visual metric indicative of the change in vision of the user.

14. The system of claim 1, wherein the guessing rate is based on wrong answers and unknown answers corresponding to a set of previously displayed optotypes.

15. The system of claim 1, wherein the machine learning model comprises at least one convolutional neural network (CNN) and the second visual metric comprises a likelihood of a presence of one or more scotoma and/or one or more metamorphopsia.

16. A method for detecting a change in vision of a user, the method comprising:
causing an optotype to be displayed on a device of the user, the optotype oriented in a first direction;
receiving acuity results corresponding to the optotype and first user input indicative a second direction associated with the optotype;
generating a first visual metric corresponding to the user based on the first direction, the acuity results, and one or more of a distance between the user and the device or a guessing rate;
causing an Amsler grid to be displayed on the device of the user;
receiving first Amsler results comprising second user input corresponding to one or more grid markings on the Amsler grid indicative of vision distortions of the user;
training a machine learning model to generate at least one visual metric corresponding to Amsler results;
generating a second visual metric of the user based on the first Amsler results and using the machine learning model;
causing a video game to be displayed on the device; and
generating a third visual metric corresponding to the user based on a user's performance in the video game.

17. The method of claim 16, further comprising generating video game metrics corresponding to the video game based on the first visual metric and the second visual metric, the video game metrics corresponding to at least a difficulty level of the video game.

18. The method of claim 16, further comprising generating a fourth visual metric corresponding to the user based on the first visual metric, the second visual metric, and the third visual metric, the fourth visual metric indicative of the change in vision of the user.

19. The method of claim 18, further comprising generating an alert based on one or more of the first visual metric, the second visual metric, the third visual metric, or the fourth visual metric.

20. The method of claim 16, wherein the video game is configured to be displayed on the device.

21. The method of claim 16, further comprising determining a test frequency, wherein one or more of the optotype or the Amsler grid is caused to be displayed on the device based on the test frequency.

22. The method of claim 16, wherein the guessing rate is based on wrong answers and unknown answers corresponding to a set of previously displayed optotypes.

23. The method of claim 16, further comprising calculating the distance between the user and the device based on a distance between two landmarks identified on an image of the user generated by the device.

24. The method of claim 16, wherein the machine learning model comprises at least one convolutional neural network (CNN) and the second visual metric comprises a likelihood of a presence of one or more scotoma and/or one or more metamorphopsia.

25. A system for detecting a change in vision of a user, the system comprising:
memory configured to store computer-executable instructions; and
at least one computer processor configured to access memory and execute the computer-executable instructions to:
cause an optotype to be displayed on a device of the user, the optotype oriented in a first direction;
receive acuity results corresponding to the optotype and first user input indicative a second direction associated with the optotype;
generate a first visual metric corresponding to the user based on the first direction, the acuity results, and one or more of a distance between the user and the device or a guessing rate;
cause an Amsler grid to be displayed on the device of the user;
receive first Amsler results comprising second user input corresponding to one or more grid markings on the Amsler grid indicative of vision distortions of the user;
train a machine learning model to generate at least one visual metric corresponding to Amsler results;
generate a second visual metric of the user based on the first Amsler results and using the machine learning model;
cause a video game to be displayed on the device; and
generate a third visual metric corresponding to the user based on a user's performance in the video game.

26. The system of claim 25, wherein the at least one computer processor is further configured to access memory and execute the computer-executable instructions to generate video game metrics corresponding to the video game based on the first visual metric and the second visual metric, the video game metrics corresponding to at least a difficulty level of the video game.

27. The system of claim 25, wherein the at least one computer processor is further configured to access memory and execute the computer-executable instructions to generate a fourth visual metric corresponding to the user based on the first visual metric, the second visual metric, and the third visual metric, the fourth visual metric indicative of the change in vision of the user.

28. The system of claim 25, wherein the at least one computer processor is further configured to access memory and execute the computer-executable instructions to generate an alert based on one or more of the first visual metric, the second visual metric, the third visual metric, or the fourth visual metric.

29. The system of claim 25, wherein the at least one computer processor is further configured to access memory and execute the computer-executable instructions to determine a test frequency, wherein one or more of the optotype or the Amsler grid is caused to be displayed on the device based on the test frequency.

30. The system of claim 25, wherein the machine learning model comprises at least one convolutional neural network (CNN) and the second visual metric comprises a likelihood of a presence of one or more scotoma and/or one or more metamorphopsia.
